(19) United States
(12) Patent Application Publication
(10) Pub. No.: US 2001/0036647 A1
(43) Pub. Date: Nov. 1, 2001

(54) FUNCTIONALLY ASSEMBLED ANTIGEN-SPECIFIC INTACT RECOMBINANT ANTIBODY AND A METHOD FOR PRODUCTION THEREOF

(76) Inventors: PRABHAKARA V. CHOUDARY, DAVIS, CA (US); ABIODUN A. OGUNJIMI, SCARBOROUGH (CA); JOHN M. CHANDLER, DAVIS, CA (US)

Correspondence Address: HANA VERNY, PETERS VERNY JONES AND BIKSA LLP, 385 SHERMAN AVENUE SUITE 6, PALO ALTO, CA, 94306

(21) Appl. No.: 09/425,075

(22) Filed: Oct. 21, 1999

Related U.S. Application Data
(60) Provisional application No. 60/105,259, filed on Oct. 22, 1998.

(51) Int. Cl. C12P 21/06; C07H 21/04; A61K 39/395; C12P 21/04; C12N 1/14; C12N 1/16; C12N 1/18; C12N 15/00; C12N 15/09; C12N 15/63; C12N 15/70; C12N 15/74; C12N 5/06; C12N 5/16

(52) U.S. Cl. 435/69.1; 435/69.6; 435/326; 435/254.11; 435/255.5; 424/130.1; 536/23.53; 435/320.1

(57) ABSTRACT

Functionally assembled antigen-specific intact recombinant monoclonal antibody produced by transformation of the methylotropic yeast, P. pastoris with mouse/human immunoglobulin genes encoding heavy and light chains. A method for production of the intact monoclonal antibodies, a recombinant yeast expression vector and the antibody-specific MRNA synthesis. A process for a large-scale production of the functionally assembled intact recombinant antibody.

This application is based on and claims priority of the Provisional Application Ser. No. 60/105,259 filed on Oct. 22, 1998.

### BACKGROUND OF THE INVENTION

1. Field of Invention

This invention concerns functionally assembled antigen-specific intact recombinant monoclonal antibody produced by transformation of the methylotropic yeast, Pichia pastoris transformed with immunoglobulin (Ig) genes. In particular, this invention concerns production of immunologically active antigen-specific intact recombinant mammalian, including human, antibody, transformed with immunoglobulin genes. The invention also concerns a method and process for production of the intact monoclonal antibody, a recombinant yeast expression vector and the antigen-specific antibody synthesis. The invention further concerns a method for large-scale production of the functionally assembled intact recombinant mammalian, including human, antibody.

### BACKGROUND AND RELATED DISCLOSURES

Recombinant DNA technology has facilitated humanization of murine monoclonal antibodies (Ann. Allergy Asthma Immunol.,81:105, (1998)) and heterologous production of antibody fragments (Res. Immunol., 149:587,(1998)). As a result, over the past 15 years, numerous antibody fragments, such as Fab, 30 FV, scFv, or diabodies, have been produced in bacterial hosts (Curr. Opinion Microbiol., 5:256, (1993)).

Prokaryotes, however, are incapable of producing complex multimeric glycoproteins, such as intact antibodies, which require posttranslational modifications in a functionally assembled form. Prokaryotes also tend to accumulate over-expressed recombinant proteins as insoluble inclusion bodies, necessitating additional denaturation-renaturation steps for recovering recombinant proteins. These steps often impair the biological function of these recombinant proteins. As an alternative, several eukaryotic hosts have been evaluated for ability to produce functionally assembled intact antibodies (New Frontiers in Agrochemical Immunology, 171-186, D. A. Kurtz et al, AOAC International, Arlington, Va. (1995)).

Mammalian cell lines have been previously investigated with some degree of success as hosts for recombinant antibody production. However, with their slow doubling rate of 24 hours or more and relatively high cost of maintenance due to more stringent sterility and growth requirements, compounded by the concerns that most of them are transformed cell lines, such mammalian cell lines have not become the hosts of choice.

Insect cell lines, infected with recombinant baculoviruses expressing antibody genes have also been tested with some success, but despite having an efficient signal sequence, about 50% of the total product has been found to be retained within the cell. The use of insect larvae, which have been demonstrated to be high producers of recombinant intact antibodies, have been limited due to concerns about potential contamination with bacterial endotoxin beyond acceptable levels. The problems described above have created a strong need for alternative methods using, preferably, other eukaryotic host(s) for large-scale production of intact antibodies and for consequent reduction of traditional dependence on animals as sole source of antibodies (Ibid, 1995).

It would, therefore, be advantageous to provide some other biological system(s) capable of producing intact antibodies which would be practical, economical, faster and safer than these systems discussed above.

Yeast has a long history as a favorite host for recombinant protein production, because of the unique advantages it offers as a unicellular eukaryote. Traditionally, the baker's yeast, Saccharomyces cerevisiae, was found to be suitable and is used as host for expression of recombinant proteins (Biotechnology, 9:1067 (1991)) including antibodies and Fab fragments PNAS USA, 85:8678 (1988)). However, despite some initial successes, it has not been possible to harness the full potential of S. cerevisiae for secreted production of intact antibodies (Nature Biotechnology, 16:773, (1998)).

In recent years, the methylotrophic yeast, Pichia pastoris (P. pastoris) has emerged as a popular host for overproduction of both intracellular and extracellular recombinant proteins, including antibody fragments (J. Biochem., 121:831, (1997); and Bio/Technology, 13:255, (1995)).

Dioxins (halogenated dibenzodioxins) are highly persistent environmental contaminants with a broad spectrum of serious health effects, and there is a strong need for accurate detection of these toxicants (Nature, 375:353, (1995)). Since large quantities of antibodies are required for immunoassay in general and for rapid detection of dioxins in particular, the feasibility of using P. pastoris for producing functional, intact antibody against the prototypical dioxin, i.e., against 2,3,7,8-tetrachloro-dibenzo-p-dioxin (TCDD) was investigated and the current method for production of large quantities of hapten-specific antibodies secreted by P. pastoris was discovered.

It is, therefore, a subject of this invention to provide an antigen-specific intact recombinant antibody that is functional and a method for large scale production of such antibody. For that purpose, P. pastoris was evaluated as a host for efficient production of a recombinant monoclonal antibody (mAb), and large quantities of the intact recombinant antibody with binding-specificity to dioxin, as a model, were produced.

The invention, therefore, concerns a large scale production and efficient secretion of a functionally assembled antigen-specific immunologically active intact recombinant antibody with binding specificity to the antigen of interest by P. pastoris.

All patents, patent applications and publications cited herein are hereby incorporated by reference.

### SUMMARY

One aspect of the current invention is a functionally assembled antigen-specific, immunologically active intact recombinant antibody produced by transformation of the methylotropic yeast, P. pastoris, with human, mouse or other mammalian immunoglobulin genes.

Another aspect of the current invention is a method for production of functionally assembled antigen-specific intact recombinant antibody by transformation of P. pastoris with human, mouse or other mammalian immunoglobulin genes.

Still another aspect of the current invention is the P. pastoris integrative expression vector (pPICZα) into which particular antibody clones are subcloned in a two-step process and the plasmid pPICZαDD1 (for anti-dioxin antibody) or other pPICZα recombinant (depending on the antigen), as desired, is produced.

Still another aspect of the current invention is the transformation of E. coli XL1-Blue with the recombinant plasmid of pPICZαDD1 or with other pPICZα recombinants.

### BRIEF DESCRIPTION OF DRAWINGS

FIG. 1 is a schematic representation of expression cassettes of the plasmid pPICZαDD1. The expression cassettes of the 666-bp chain (L-chain) and 1332-bp heavy chain (H-chain) genes were each fused to the 267-bp yeast α-factor signal sequence (ss), under the control of the yeast promoter (AOX1-P). A yeast transcription sequence (TT) marks the 3′ end of each expression cassette. The restricted enzyme sites used in the construction of the plasmid are indicated.

FIG. 2 is a PCR analysis of P. pastoris transformants for pPICZαDD1 genomic integrates. PCR products of P. pastoris transformants using primers specific for AOX1 and for the antibody light chain and heavy chain were analyzed using agarose electrophoresis.

FIG. 3 is Northern blot analysis of RNA-blots of transcripts from clones 11501-1 (DD1) and 112535-1 (DD1) and two controls (ve+) and (ve−). The blots were probed with ³²p-labeled PCR amplicon of the antibody light chain and detected by autoradiography.

FIG. 4 is Western blot analysis of culture media and cell lysates of recombinant yeasts probed with AP-goat anti-mouse IgG and visualized by AP color reaction.

FIG. 5 is a graphic depiction of ELISA result demonstrating recombinant antibody binding and specificity to dioxin (hapten). The antibody-hapten binding was measured directly in an ELISA.

FIG. 6 shows kinetics of antibody produced in yeast cells or secreted into cultured media, as revealed by immunoblots of cell lysates, and culture supernatants of P. pastoris probed with AP-goat anti-mouse IgG.

### DEFINITIONS

As used herein:

“Pichia pastoris” or “P. pastoris” means a methylotropic yeast, a single-celled microorganism that prefers aerobic growth and can be grown to much higher cell densities than fermentative yeasts.

“DD1” means mouse hybridoma secreting antidioxin monoclonal antibody described in the U.S. Pat. No. 5,334,528.

“Antibody genes” means and is used to denote the mRNA, cDNA, or genomic or chemically synthesized DNA fragments coding for an antibody.

### DETAILED DESCRIPTION OF THE INVENTION

This invention concerns and describes a novel method for production of functionally assembled antigen-specific, immunologically active intact recombinant antibodies. The method is generally useful for preparation of any kind of antibody and is also suitable for large-scale production of human and other mammalian antibodies. The method is easy, practical, fast and safe.

I. Method for Production of Functionally Assembled Antigen-Specific Intact Antibody

The method of the invention for production of functionally assembled antigen-specific intact monoclonal antibody, using transformation of P. pastoris, has a general utility and essentially any antibody can be produced or secreted by P. pastoris as long as the yeast expression vector carrying antibody genes can be appropriately assembled.

Briefly, P. pastoris is transformed with human, mouse or other mammalian immunoglobulin genes encoding heavy (gamma) and light (kappa or lambda) chains of antigen-specific antibody. Antibody genes are isolated from a hybridoma that recognizes certain specific antigen and the transformed yeast clones generated according to the method of invention then specifically recognize that particular antigen. Following the transformation, P. pastoris produces and secretes large amounts of a functionally assembled antigen-specific intact monoclonal antibody into the culture supernatant.

In a more detailed description of the method, a recombinant yeast expression vector (pPICZα) with dual expression cassettes is constructed, each cassette carrying the inducible alcohol oxidase (AOX1) promoter, fused to the Saccharomyces cerevisiae α-factor signal sequence. P. pastoris is then transformed with these constructs, and the resulting transformant secretes functionally assembled intact recombinant antibody molecules into the medium from where it is readily recovered using affinity purification procedures.

Specificity of the produced antibody is determined by demonstrating the antibody-specific mRNA synthesis in recombinant yeast using Northern blot analysis. When the specific antibody is produced, immunoblot and ELISA analyses of concentrated culture supernatants harvested a few days post-transformation reveal the presence of antigen-specific human, mouse or other mammalian species-specific immunoglobulins. Assaying of the culture supernatants by ELISA then shows specific binding activity to the specific antigen against which the antibody is raised or to a cross-reactive congener. The binding affinity of the produced recombinant IgG is the same as, and/or comparable to, that of the parent IgG.

II. General Method for Production of Antigen-Specific Intact Antibodies on Large Scale

The method according to the invention utilizing P. pastoris-based expression system was found suitable for a large-scale production of intact recombinant antibodies.

A typical process for large scale production of antigen-specific antibodies according to the invention comprises following steps:

Step 1. Selection and identification of the antigen against which the antibody is to be raised.

Such antigen selection is entirely need-based. If the aim is, for example, to produce an antibody against the AIDS virus, HIV-1, then inactivated HIV-1 is used as the immunogen. If the genes (or their cDNA) are available that code for anti-HIV-1 antibody, the method proceeds directly to step 2 of the procedure and those genes are expressed using the vector system and method or an appropriate variation thereof of the invention. If the aim is, for example, to produce antibodies that recognize the malaria antigen (Ag), then the anti-malaria Ag antibody genes, if available, are used or these genes are isolated from a hybridoma secreting monoclonal antibodies that recognize and bind malaria antigen.

In yet another example of how to obtain the antibody, an animal is immunized with the target immunogen or carrier-conjugated hapten, a cDNA library of either the IgG repertoire or the entire mRNA component is generated and the library is screened for clones with specific recognition of the target antigen. Similarly, the antibody genes may be isolated directly from a previously exposed or a naive animal or human and expressed using the expression system of the invention.

In the case of an immunogen/hapten that is a chemical compound, it is either purchased or custom-ordered from a chemical company (e.g., Aldrich, Chemicon) or, if it is not available commercially, it is synthesized in laboratory. Likewise, protein or peptide immunogens/antigens can either be purchased from any of a number of biochemical companies (e.g., Sigma Calbiochem, etc.), purified from target source, such as animal tissues/cells, plants, bacteria, viruses, etc., prepared from an existing precursor, such as peptides from a pre pro- or pro- or mature protein, or obtained by recombinant methods. Haptens and peptides that are too small to be effective as immunogens are used in conjunction with suitable carriers for production of antibody, generally monoclonal antibody, and the antibody-encoding genes are isolated from the antibody-producing cells and used in the Pichia expression system for creating a source of candidate antibody as well as antibody genes.

Step 2. Isolation or chemical synthesis or PCR-/T7-amplification of antibody-encoding CDNA, mRNA or genomic DNA fragments.

Methods for isolation and chemical synthesis or PCR or T7 amplification of cDNA, MRNA or genomic DNA are known in the art. Any and all these methods may be used for the step 2 of this method.

Step 3. Assembling the antibody genes into expression cassettes.

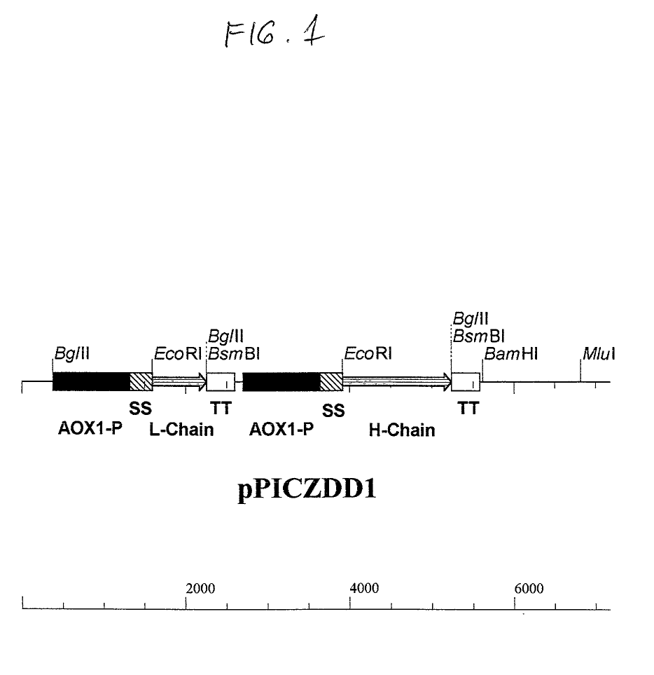

Assembling the antibody genes into expression cassettes, as seen in FIG. 1 is achieved by, for example, subcloning the light and heavy chain cDNAs in tandem as EcoRI-BglII/BSmBI fragments each flanked by a signal sequence, such as, for example yeast α-factor, preceded by a yeast promoter, such as for example alcohol oxidase AOX1-P, at the 5′-terminus and by the yeast transcription termination sequence at the 3′ terminus. Possible variations of this step include use of antibody genes such as the use of the entire light chain (kappa or lambda), partial use of light chain comprising only one, two or three CDRs or parts thereof, one or two CDRs in combination with part or complete framework region (homologous, heterologous or non-Ig but neutral compatible sequence from a diverse source such as myoglobin, actin or a synthetic peptide of unrelated origin and/or function) and/or a heavy chain gene of similar variations. Other variations include use of signal and promoter sequences, including but not limited to those obtained from insects, yeasts, bacteria, viruses, mammals, and plants, as long as they are functional in P. pastoris.

Step 4. Preparing a yeast expression vector pPICZα for cloning of antibody genes.

A yeast expression vector, for example, pPICZα is prepared for cloning of antibody genes, for example, by restriction digestion with EcoRI and BamHI. Other restriction enzymes unique to the vector or such that if they were present in the antibody genes they can be repaired to restore the functional integrity of the antibody genes in the recombinant plasmid may be also used for preparation of the vector.

Step 5. Cloning of antibody gene expression cassettes into the Pichia expression vector (pPICZα).

Antibody gene expression cassettes are cloned into the Pichia expression vector (pPICZα) to generate recombinant plasmid (pPICZαLH) or a variant thereof using methods known in the art for cloning.

Step 6. Transforming bacteria with recombinant plasmid pPICZαLH or its variant.

Bacteria, for example Saccharomyces cerevisiae are transformed with recombinant plasmid pPICZαLH or its variant using methods known in the art.

Step 7. Amplifying and isolating the recombinant plasmid on preparative scale.

Amplification and isolation of the recombinant plasmid on preparative scale is achieved by using standard methods of large-scale growth of the recombinant and plasmid isolation such as the alkaline lysis method described in Current Protocols in Molecular Biology, Ausubel E. M. et al., Wiley-Interscience, New York, (1990).

Step 8. Preparing and transforming P. pastoris spheroplasts.

P. pastoris spheroplasts are transformed with BglIII-linearized, or in alternative NotI, SacI, SalI and Stul-linearized recombinant plasmid, resulting in in vivo homologous recombination replacement of the yeast chromosomal AOX1 sequence with the 5′ AOX1-antibody gene cassette of the recombinant plasmid.

Step 9. Selectively growing the recombinants and eliminating the non-recombinants.

Selective growth of recombinants and elimination of non-recombinants is achieved by plating transformants on medium containing zeocin (100 ug/ml). Zeocin may be replaced by other compounds. Such replacement depends on the selection marker (gene) included in the plasmid. Some examples of possible replacement include, but are not limited to, G418, trimethoprim, and drugs/compounds/polypeptides that limit the growth of wild type P. pastoris in contrast to the yeast that is transformed with a plasmid containing said selection gene(s).

Step 10. Screening of the yeast transformant colonies for antibody expression.

Screening of the yeast transformant colonies for antibody expression is achieved by colony-immunoblotting for the origin of the recombinant proteins (antibody): human/mouse/or other; antigen-/hapten-binding activity thereby providing preliminary identification of putative positive clones. In alternative, any other means of distinguishing the recombinant over the host background may be used.

Step 11. Analyzing the putative positive yeast clones for chromosomal integrates of the expression cassettes.

The putative positive yeast clones are analyzed, for example, by PCR or by restriction analysis, for chromosomal integrates of the expression cassettes of both light and heavy chain cDNAs at the correct locus.

Step 12. Performing a Mut⁺/Mut^{s} test for selecting +His +Mut⁺phenotypes.

Performing a mut⁺/Mut^{s} test by replica-plating transformant colonies on (i) a −His +glucose plate and on (ii) a −His +methanol plate and, because Mut⁺colonies are slow utilizers of methanol while Mut+ colonies are normal, that is relatively rapid, utilizers of methanol facilitated by AOX1 promoter, selecting +His +Mut⁺phenotypes.

Step 13. Confirming the DNA insert/junction sequence integrity.

The DNA insert/junction sequence integrity is confirmed by nucleotide sequence analysis using standard methods of DNA sequencing, such as the chemical sequencing method or the dideoxy termination method including the automated methods (Current Protocols in Molecular Biology, Ausubel F. M. et al., Wiley-Interscience, New York, (1990)).

Step 14. Inducing recombinant antibody expression and growth.

Inducing recombinant antibody expression and growth by, for example, methanol [0.5 to pb 1.5%, v/v] and glycerol [1%, v/v))at 30° C. or any other conditions including potential gratuitous inducers and other growth conditions that elicit induction of antibody.

Step 15. Establishing the antibody authenticity.

The antibody authenticity is established, for example, by Northern blot/RNA protection analysis of the clones.

Step 16. Detecting the presence of the recombinant antibody.

The presence of the recombinant antibody is detected, for example, by Western blot analysis of the yeast cellular proteins and proteins secreted into the culture supernatant of the yeast clones.

Step 17. Demonstrating the antibody-antigen-specific binding activity.

Specificity of recombinant antibody specific antigen-antibody binding is confirmed, for example, by ELISA or other methods that recognize antigen-antibody reaction.

Step 18. Optimizing recombinant antibody production. optionally, the production of recombinant antibody is optimized by testing a broad range of culture and induction conditions.

Step 19. Purifying and storing the recombinant antibody.

Optionally, the recombinant antibody is purified and stored under conditions that favor its optimal stability and recovery, by for example, storing the antibody in the presence of protease-inhibitors, at −80° C. or in the presence of cryoprotective agents such as 50% glycerol.

The method described above allows expression of cDNA fragments encoding antibody light and heavy chains isolated from a pre-existing hybridoma, as illustrated below in Section III, for the preparation of the anti-dioxin antibody. Antidioxin antibody and hybridomas for their expression are described in U.S. Pat. No. 5,334,528 hereby incorporated by reference for method of producing hybridomas. The anti-dioxin genes were isolated from the hybridomas DD1 or DD3 and were genetically engineered into the Pichia expression system and coordinately expressed, producing immunologically active intact recombinant antibody.

The process for the preparation of any antigen-specific antibody according to the invention is able to utilize any existing hybridoma. Once the light and heavy chain of the antibody cDNAs are isolated by one of the standard methods known in the art, the ends of the cDNA fragments are modified to match any one or more of the multiple cloning sites (BamHI, SnaBI, EcoRI, AvrII and NotI), and cloned into that site in a vector of the pPICZ family.

The same approach is useful for genomic PCR amplicons or clones coding for antibody light chain and heavy chain open reading frames (ORFs).

The process is further useful and applicable to any available mammalian hybridomas, as well as to human hybridomas, as long as appropriate primer sequences are designed or selected for sequence amplification. Moreover, once the antibody genes are cloned into a Pichia expression vector, the rest of the scheme (Steps 6 through 19) is the same as described above but for the antigen-specific reagents.

The method is generally useful also for expression of antibody genes isolated from clinically and industrially important hybridomas producing monoclonal antibodies to c-myc, Her2/neu, lymphoma, etc., or for cloning and expression of antibody genes from immunized or naive animals or humans. The only variations in the method apply to cloning of the antibody genes. Resulting clones determine the properties of the antibody that is eventually produced using this approach.

If PCR amplification, for example, is used for cloning the antibody genes, then the primer sequences used for PCR amplification determine the antibody type, form and size. In other words, the primers can be designed to isolate genes from an animal or from a human, and to produce an intact antibody or a fragment such as Fab.

If gene probes are used for isolating antibody genes, then the antibody that is produced from them is dependent on the probes used. If probes are specific for a human antibody, then the recombinant antibody that is expressed from those genes is the human antibody. On the other hand, if the probes are specific to a mouse antibody then the resulting recombinant antibody is the mouse antibody. However, by capitalizing on the relatively close homology between the mouse and human gene sequences which permits generation os so called humanized mouse, it is possible to use gene probes to isolate antibody gene clones for cross-species by simply reducing the stringency of the probe hybridization conditions.

Further, if the sequences of the primers or probes are specific, for instance against mouse c-myc antibody by recognizing unique sequences in their CDRs (complementarity determining regions) and not the framework regions (common to different antibodies), then the cloned antibody genes will code only for c-myc antibody and not any other antibody. By the same token, when the primers or probes are chosen to recognize the framework regions of the antibody chains, a heterogeneous population of antibody genes is obtained.

One precondition for the method is a selection of a single pair of heavy and light chain genes either at the time of constructing recombinant yeast expression plasmid or at the time of isolating individual yeast clones expressing recombinant antibodies. Typically, to clone antibody genes directly from an animal or a human, the genes for any antibody can be isolated and expressed. There is no other limitation within the method, except for the necessity of designing the primers or probes or screening the libraries of clones by using methods known in the art.

The procedure for producing any antibody is thus essentially the same as the one described above for general purposes, and below, as exemplarized for recombinant anti-dioxin antibody, said procedure needing only to suitably incorporate the variations described for steps 1-5.

III. Method for Production of Functionally Assembled Anti-Dioxin Specific Intact Antibody

The method of the invention was developed and tested on anti-dioxin antibody because the anti-dioxin antibody (IgG gamma and kappa) genes are readily available in the inventors laboratory and also because, as a practical matter, a large quantities of the anti-dioxin antibody was urgently needed for fast and reliable detection of dioxin contamination in the environment.

In the following description of the procedure used for production of anti-dioxin antibody according to the invention, subsections A-E deal with the specific description of the method for production of anti-dioxin antibody, including steps, procedures, materials and test results.

A. Anti-Dioxin Antibody Synthesized by P. pastoris Transformed with pPICZαDD1

The invention is based on the discovery that the methylotropic yeast P. pastoris can be transformed to secrete large quantities of a dioxin-specific antibody when transformed with Pichia expression vector carrying the genetic information for expression of anti-dioxin antibody.

1. Methylotropic Yeast Pichia pastoris

P. pastoris strain SMD1168 (pep4 his4) was identified as a suitable host for antibody production according to the invention.

P. pastoris strain SMD1168 (pep4 his4) and the P. pastoris integrative expression vector (pPICZαB) were obtained from Invitrogen (Carlsbad, Calif.).

2. Transformation of Pichia pastoris

The P. pastoris strain SMD1168 was transformed with recombinant plasmid, pPICZαDD1 and zeocin-resistant transformants were isolated.

A schematic representation of expression cassettes of pPICZαDD1 plasmid is shown in FIG. 1. The 7164-bp recombinant plasmid, pPICZαDD1, contains a bacterial origin of replication (COIEI), Zeocin-resistance gene (ZeO^{R}) for selection of both E. coli and yeast transformants, and the expression cassettes of anti-dioxin antibody light- and heavy chain genes.

The final construct was assembled by replacement-ligation of the 3134-bp (MluI-BamHI) fragment of the recombinant plasmid construct containing the light chain expression cassette, and the 4030-bp (MluI-BamHI) fragment from the heavy chain expression cassette construct.

FIG. 1 is a schematic representation of expression cassettes of the plasmid pPICZαDD1. The expression cassettes of the 666-bp light-chain (L-chain) and 1332-bp heavy-chain (H-chain) genes were each fused to the 267-bp yeast a-factor signal sequence (SS), under the control of the yeast promoter (AOX1-P). A yeast transcription termination sequence (TT) marks the 3′ end of each expression cassette. The restriction enzyme sites used in the construction of the plasmid are indicated.

DNA manipulations were performed using standard techniques as described in Current Protocols in Molecular Biology, Eds. Ausubel, F. M., et al., Wiley-Interscience, New York, (1994) or according to vendor recommendations. The light-chain (666-bp) and heavy-chain (1332-bp) sequences of the mouse hybridoma DD1 (Gene, 19:388, (1994)) secreting antidioxin mAb, have been cloned and sequenced and expressed in E.coli as Fab according to J. Agric. Food Chem., 46:3381 (1998).

The anti-dioxin antibody genes were sub-cloned into pPICZαB in a two-step process. First, the sequence encoding the light-chain (666 bp) or heavy-chain (1332 bp) mature peptide was PCR-amplified from cloned cDNA using primers designed to produce a blunt 5′ terminus and a 3′ nested BglII/XbaI site preceded by a stop codon, and a codon for cysteine (TGC) introduced before the stop codon in the heavychain sequence to facilitate conjugation of the recombinant antibody to a peptide tag for affinity purification.

The light-chain and heavy-chain amplicons were separately cloned into pPICZαB, under the control of yeast AOX1 promoter, translationally fused to S. cerevisiae a-factor signal sequence, producing pPICZα-L (light) and pPICZα-H (heavy) plasmid, respectively.

In the second step, plasmid pPICZ-L was digested with BamHI+MluI and pPICZα-H with BglII+MluI, and the 3134-bp vector fragment containing the light-chain expression cassette and the 4030-bp vector fragment containing the heavy-chain expression cassette, were gel-eluted. The final 7164-bp construct pPICZαDD1 was assembled by replacement-ligation of the gel-eluted light-chain and heavy-chain fragments.

Detailed method and transformation conditions for introduction of the pPICZαDD1 construct into E. coli are described in Example 2.

3. Screening For Antibody Expression

Screening of transformant yeast colonies for antibody expression was performed as described in Example 3.

Briefly, the zeocin-resistant yeast colonies were patched on nitrocellulose filters and grown for 2 days on induction plates at 30° C. The colony-blots were probed with AP-goat anti-mouse monoclonal antibody (Boehringer Mannheim, Indianapolis, Ind.) as recommended by the vendor.

DNA sequencing procedure is described in Example 4. DNA was processed for sequencing using the ABI Tag DyeDeoxy Terminator Cycle Sequencing kit, based on the chain terminating method. Nucleotide sequence was determined in an automated DNA Sequencer and data analyzed using the PE/ABI editing and assembly software.

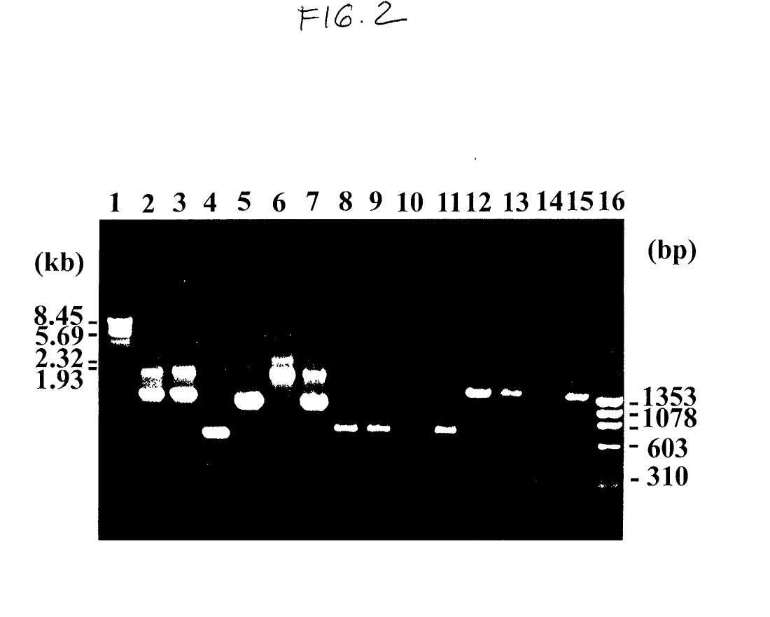

Transformants for antibody expression were screened by PCR analysis as described in Example 5. Results are shown in FIG. 2.

Screening of the transformants for antibody expression was performed by preparing a nitrocellulose membrane-replica of transformants growing on an agar plate with induction medium and probing it with anti-mouse IgG (data not shown).

Two transformants, 11505-1 (Mut^{s}) and 112535-1 (Mut⁺) that tested strongly positive by this screen were analyzed by colony-PCR using relevant primers to confirm chromosomal integration of the plasmid DNA sequences.

FIG. 2 shows PCR analysis of P. pastoris transformants for pPICZαDD1 genomic integrates. PCR products of P. pastoris transformants using primers specific for AOX1 are shown in lanes 2-7, the antibody light chain are shown in lanes 8-11 and heavy chain in lanes 12-15. Transformants were analyzed using agarose (1.5%, w/v) gel electrophoresis. FIG. 2, lane 1 shows Mr. markers (Phage lambda BstEII digest; New England BioLabs, Beverly, Mass.); lanes 2, 8, and 12 show clone 11505-1; lanes 3, 9, and 13 show clone 112535-1; lanes 4, 10, and 14 show unmodified vector (−ve control); lanes 5-7, 11, and 15 show recombinant plasmid (+ve control); lane 5 shows light-chain gene; lane 6 shows heavy-chain gene; lanes 7, 11 and 15 show vector with both light- and heavy-chain genes; lane 16 shows Mr. markers (PhiXl74 HaeII digest; New England BioLabs). Molecular weights of the markers are shown in kilobases (kb) on the left of the gel and in base pairs (bp) on the right.

Results obtained with these two representative clones, shown in FIG. 2, confirmed the integrity of expression cassettes in the 11505-1 and 112535-1 clones by DNA sequence analysis.

PCR analysis of P. pastoris transformants using primers specific for the antibody genes or for the AOXI 5′ and 3′ termini showed intact full-length light- and heavy-chain gene expression cassettes integrated in genomic DNA of the transformants. Control transformants harboring vector alone yielded no amplification products in PCR with Ab gene primers. DNA sequence analysis of the PCR products from recombinants confirmed that the primary structure of the target sequences was preserved.

4. Induction of Antibody Production

Following the confirmation and integrity of expression cassettes of the clones, the antibody production by recombinant P. pastoris clone was induced according to Example 6.

Induction of recombinant antibody expression was typically performed as follows. A P. pastoris transformant was cultured for two days with shaking at 250 rpm in BMGY broth (buffered glycerol-complex medium with yeast extract) at 30° C. The yeast cells were collected by centrifugation and transferred to the induction medium. Beginning on the second day of growth, methanol was added daily to a concentration of 0.5% (v/v), to induce the AOX1 promoter-driven production of recombinant antibody.

Screening of the recombinants for antibody expression was performed using the colony-blot assay (data not shown). The method involved making a nitrocellulose membrane-replica of recombinants on an agar plate with induction medium and probing it with AP-goat anti-mouse IgG, after gently washing the cell-debris off with non-fat milk (5%, w/v, in TBST).

Fifteen recombinants showing a high degree of reactivity, indicating potential for high levels of recombinant Ab production, were picked for further analysis. From among those that tested positive by colony-PCR, diagnostic restriction digestion and DNA sequence analyses identified two clones as clones 11505-2 and 112535-1. These two clones were chosen for further characterization.

Specificity of transcripts was determined by Northern blot analysis according to Example 7.

For Northern blotting, total RNA was extracted from both clones (11505-1 and 112535-1) and the vector control, each induced in a 5-ml culture in MMH medium at 30° C. for 4 days.

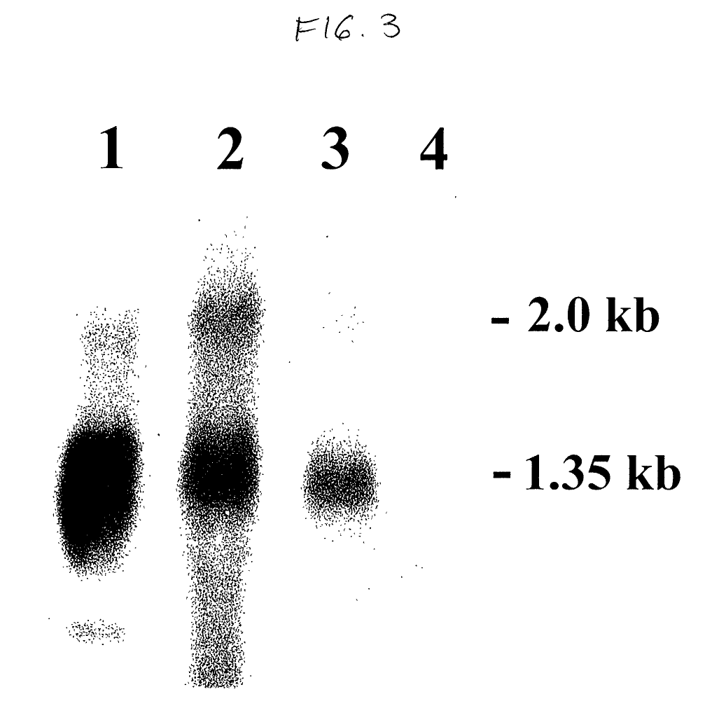

RNA (20 μg sample⁻¹) was denatured, resolved by 1% agarose gel electrophoresis in 1×MOPS buffer containing formaldehyde 1.2%), transferred to a nylon membrane (Hybond-N; Amersham Pharmacia Biotech, Piscataway, N.J.) and probed with ³²P-labelled light-chain amplicon. The blot was washed, air-dried and exposed to a Kodak X-O-matic film for 24 hour at −80° C. Results are shown in FIG. 3.

FIG. 3 is Northern blot analysis of total RNA transcripts from the two (11505-1 and 112535-1) clones and two controls (one positive and one negative) probed with ³²P-labelled PCR amplicon of the antibody light chain and detected by autoradiography. Lane 1 shows a light-chain amplicon (+ve control), lane 2 shows clone 11505-1; lane 3 shows clone 112535-1; lane 4 is a vector (−ve control). The RNA Ladder (New England BioLabs, Beverly, Mass.) stained with ethidium bromide was used as reference for estimating RNA sizes.

Transcripts detected in the Northern blot as seen in FIG. 3 were specific and corresponded to the sizes expected for both light- and heavy chains. Although the probe was derived from the light chain, it also recognized the heavy-chain transcript, because of the partial homology shared between the two chains. The variation observed in the strength of signals produced in Northern blot by different samples reflected the difference in relative levels of antibody expressed by the clones analyzed. By this measure, clone 11505-1 (lane 2) expressing the DD1 antibody showed greater expression levels than 112535-1 (lane 3).

As seen in FIG. 3, Northern blots of total RNA from induced (96 hours) cultures of the clones 11505-1 (lane 2) and 112535-1 (lane 3), probed with gel-purified and labeled light-chain gene, showed specific transcripts of 1360 bp and 2022, corresponding to the sizes expected for the light- and heavy-chain genes, respectively.

B. Efficiency of Intact Antibody Secretion by Pichia Pastoria

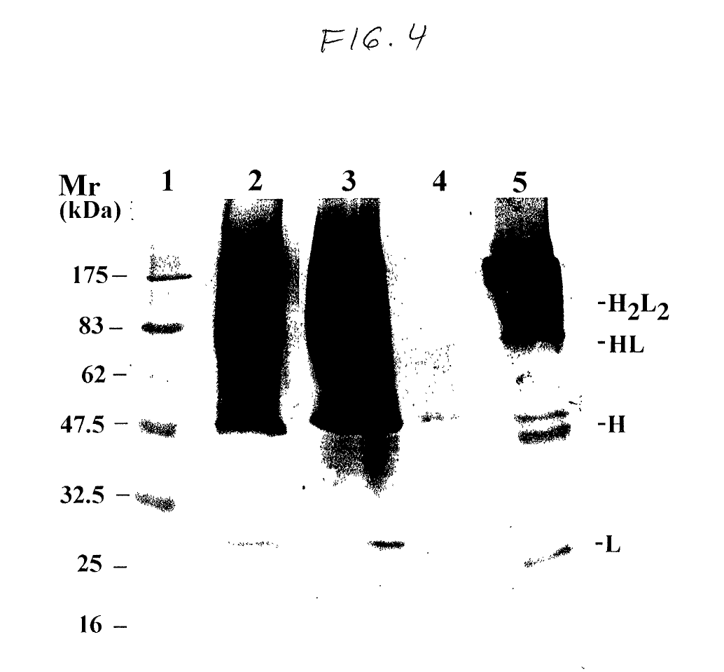

The intactness of the inserts and the accuracy of the junction sequences were confirmed using PCR procedure described in Example 9 and by nucleotide sequence analysis. For Western immunoblot analysis, cell lysates and 25×concentrated media containing between 50-75 μg a total protein sample⁻¹, were resolved by non-reducing 10% SDS-PAGE using Tris-glycine SDS buffer and electroblotted onto a PVDF membrane (Millipore, Bedford, Mass.). The blot was processed and probed with AP-goat anti-mouse IgG (1:5000, in TBST, pH 8.0) using BCIP and NBT. Results are shown in FIG. 4.

FIG. 4 shows Western blot analysis of culture media and cell lysates of recombinant yeasts. Nitrocellulose blots containing equivalent amounts of total yeast proteins from culture media and cell lysates from P. pastoris clones and controls were probed using AP-goat anti-mouse IgG and were visualized by AP color reaction. For FIG. 4, H₂L₂ shows intact Ab, HL is a heavy chain-light chain monomer; H is gamma (heavy) chain; L is kappa (light) chain. Lane 1 shows pre-stained protein Mr markers (New England BioLabs) with sizes indicated on the left of the gel, lane 2 shows clone 11505-1; lane 3 shows clone 112535-1, lane 4 shows vector (−ve) control and lane 5 shows mouse IgG, 0.25 μg (+ve control).

Western-blot analysis of culture media of the clones 11505-1 and 112535-1 revealed mouse antibody chains with the sizes expected for monomers of light chain (25 kDa), heavy chain (50 kDa), intact antibody (150 kDa) and some intermediate assemblages as seen in FIG. 4, indicating proper assembly of antibody molecules. Results of nonreducing SDS-PAGE analysis of culture media and cell lysates of the clones 11505-1 and 112535-1 (data not shown) corroborated immunoblotting results seen in FIG. 4.

Antibody levels in cell lysates of both clones (11505-1 and 112535-1), at approximately 10% of the total product, as assessed by Western blotting, were consistently lower than those found in culture medium, demonstrating that a major portion, approximately 90% of the antibody produced by recombinant P. pastoris was secreted into the supernatant. These results show that P. pastoris secretes intact antibody very efficiently. Non-reducing SDS-polyacrylamide gel electrophoresis (SDS-PAGE) of culture media and cell lysates showed protein bands migrating at positions expected for the light (25 kDa) and heavy chain (160 kDa) assembled antibody.

C. Specificity of the Recombinant Antibody

The binding specificity of the recombinant antibody to its cognate hapten, in this case dioxin, was evaluated using two independent approaches, slot-blot and ELISA described in Examples 9 and 10.

The hapten-binding activity of recombinant antibody was assayed using ELISA. Briefly, 96-well microtiter plates (MaxiSorp Nunc-Immuno Plate, Nalge Nunc International, Denmark) coated with 10-50 ng range of BSA-dioxin, trans-3(2,3,7, 8-tetrachlorodibenzo-p-dioxin-1-yl) propenoic acid, in 100 μl well⁻¹ of 50 mM bicarbonate buffer, pH 9.6, were incubated with 100 μl of a 1:10 dilution (in TBST) of culture medium from an induced culture of each recombinant and were probed with HRP-goat anti-mouse IgG (Pierce Chemical, Rockford, Ill.) using the chromogenic TMB Microwell Peroxidase Substrate System (KPL, Gaithersburg, Md.). The plates were read at 450 nm, using the UVmax kinetic microplate reader (Molecular Devices, Menlo Park, Calif.), and the readings were analyzed using the software package Softmax (Molecular Devices, Menlo Park, Calif.).

The hapten-binding activity of the recombinant antibody was analyzed using slot-immunoblotting. All incubations were performed at 26-28° C. The hapten (BSA-dioxin, diluted 1:3600) was spotted in 10 ul volumes (to give 250 ng slot⁻¹) on a nitrocelulose membrane (S&S, Keene, N.H.), using a slot-blot apparatus (Life Technologies, Rockville, Md.). The blot was allowed to air-dry, blocked with non-fat milk (10%, w/v, in TBST) for one hour, and incubated with gentle shaking (120 rpm) on an orbital shaker (Lab-Line, Melrose Park, Ill.) for one hour with 2 ml of 1:10 dilution in TBST of the medium from induced cultures of the clones. The blot was washed, incubated with AP-goat anti-mouse IgG and developed as described for the Western immunoblot.

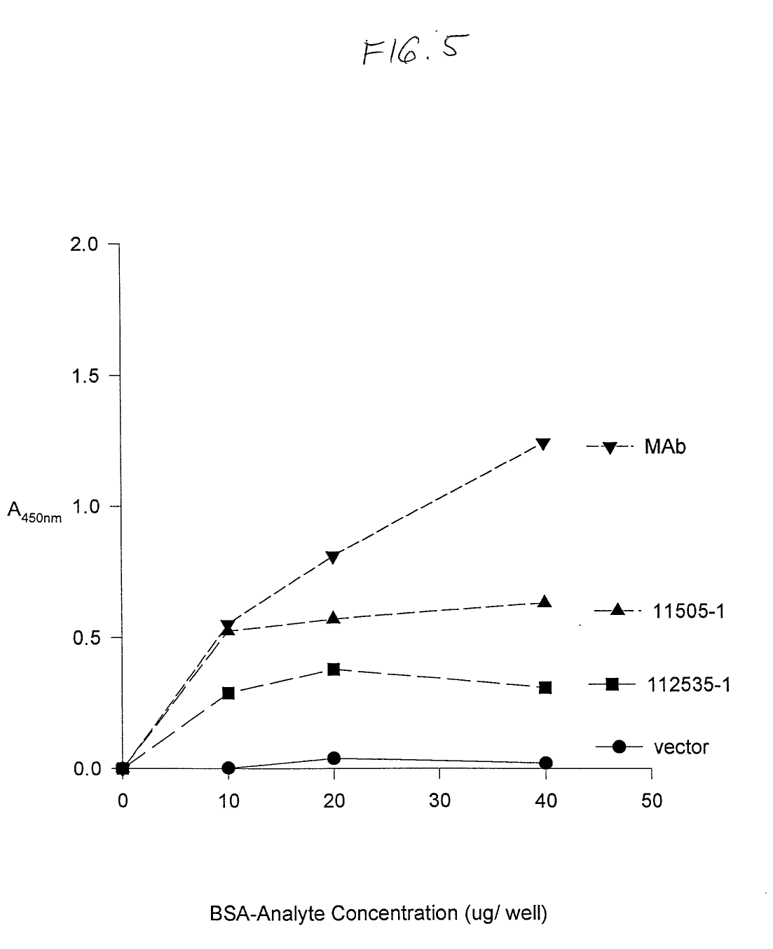

Both approaches demonstrated specific binding of the recombinant antibody to cognate hapten, dioxin, with affinities comparable to those of the parental mAb, DD1. These values can be affirmed only after precise quantification, of protein concentrations of the recombinant antibody after accounting for the Pichia cellular proteins secreted into medium. Results are seen in FIG. 5.

FIG. 5 is a graphic depiction of ELISA result demonstrating recombinant antibody binding to dioxin. The antibody-hapten binding was measured directly by ELISA. The coating hapten, BSA-dioxin (10 to 40 ng well⁻¹) in a 96-well microtiter plate was incubated with serial dilutions of culture medium from clones 11505-1 and 112535-1, vector (−ve) control, or DD1 mAb (+ve control), and probed with HRP-goat anti-mouse IgG using TMB. The A₄₅₀ readings indicate the hapten-binding activity of the recombinant antibody. Results shown in FIG. 5 demonstrate that the produced recombinant antibody is functional.

D. Kinetics of Monoclonal Antibody Production

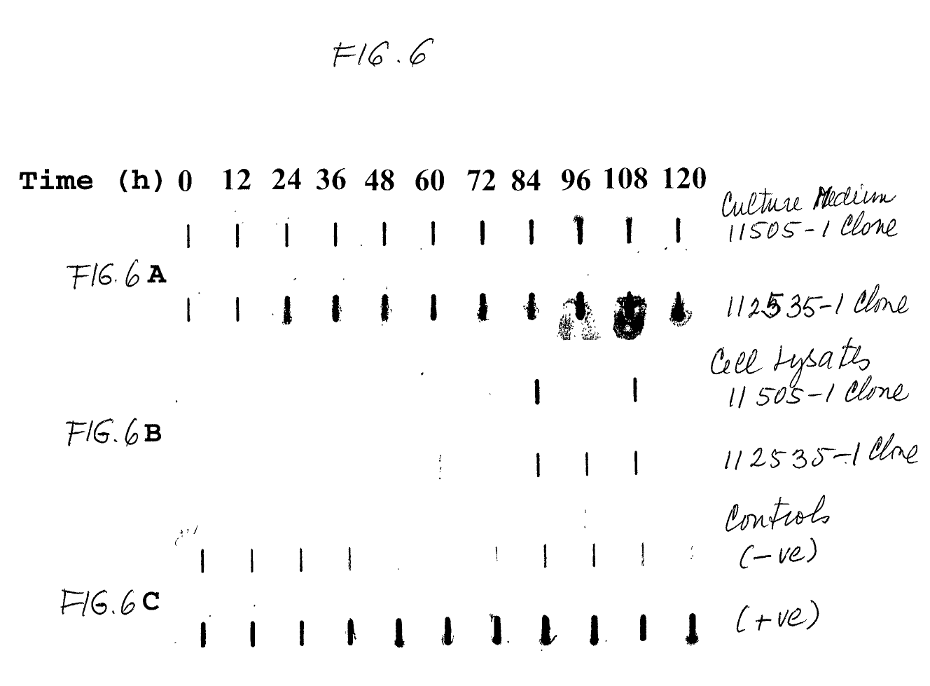

Studies performed to determine the kinetics of monoclonal antibody have shown that synthesis and secretion of antibody are optimal between 72 and 108 hours. Detailed procedure is described in Example 10. Results are shown in FIG. 6.

FIG. 6 illustrates kinetics of antibody production or secretion. Slot-blots of immobilized cell lysates or culture media from the clone cultures harvested in 12-hour intervals of induction (12 to 120 hours) were probed with AP-goat anti-mouse IgG. The clones and the duration of induction, in hours, are indicated. FIG. 6A shows culture media (supernatant), where top row shows clone 11505-1 and bottom row shows clone 112535-1. FIG. 6B shows cell lysates, where top row shows clone 11505-1 and bottom row shows clone 112535-1. FIG. 6C top row shows vector (−ve) control and bottom row shows mouse IgG (+ve control).

The kinetics of antibody production/secretion were followed by withdrawing portions of the culture at various intervals of methanol-induction of the clones and determining the antibody levels by slot-immunoblot analysis. As seen in FIG. 6, anti-dioxin antibody was detectable in culture medium between 12 hours and 120 hours of induction, with highest levels of about 10 to 36 mg l⁻¹ detected between 72 and 108 hours.

Although these levels are lower than those reported for other recombinant proteins or for antibody fragments (200 mg l⁻¹), they are the highest and set the highest range ever obtained for any intact antibody, which is a more complex multimeric glycoprotein, than the molecules previously reported. These levels can be further augmented by using fermentation approaches.

### UTILITY

This invention provides a method of general utility for production of large quantities of any antigen-specific antibody using modified yeast organism. Using the method of invention, the large quantity of compound specific and defined monoclonal antibody is produced without the necessity of immunizing and recovering and purifying antibodies and/or other lengthy procedures. The method is practical, economical, easy, safe and fast and in about three days, the monoclonal antibody is produced by the transformed yeast if the vector and expression vehicles for transformation are available or are prepared according to the invention.

The above described findings demonstrate the suitability of P. pastoris expression system for both small and large-scale production of functional, antigen-specific intact antibodies. The recombinant antibodies produced by the method of the invention are useful, for example, for immunodiagnostic and immunotherapeutic purposes. Since recombinant proteins produced in P. pastoris lack terminal α 1,3 glycan linkages responsible for hyper-immunogenicity, the antibodies produced in P. pastoris are particularly suitable for therapeutic applications.

Functional assembly of antibodies produced in P. pastoris also suggests the potential of P. pastoris for construction of antibody libraries and screening them with any antigen of interest using colony-immunoblotting.

### EXAMPLES

### Materials

Restriction enzymes were purchased from New England BioLabs (Beverly, Mass.), and Taq Polymerage from Promega (Madison, Wis.).

cDNA clones of the heavy and light chains of the antidioxin mouse mAbs, DD1 and DD3, were a gift from A. Recinos III and L. Stanker. Primers, designed on the basis of the nucleotide sequence of the above cDNAs, were synthesized by Life Technologies (Gaithersburg, Md.).

96-well microtiter plates (Nunc-Immunoplate, Maxisorp) were from Nalge Nunc International (Roskilde, Denmark), and HRP-goat anti-mouse IgG was obtained from Pierce Chemical (Rockford, Ill.). All chemicals were of reagent grade from Fisher Scientific (Pittsburgh, Pa.) or from Sigma (St. Louis, Mo.).

### Example 1

### Microbial Strains and Culture Conditions

This example identifies microbial strains and culture conditions used for the purposes of this invention.

Escherichia coli strain XL 1-Blue was used as host for plasmid amplification, using YB broth (1.5% tryptone, 1% yeast extract, 0.5% NaCl). P. pastoris SMD1168 (pep4 his4) and the yeast expression vector (pPICZαB) were obtained from Invitrogen (Carlsbad, Calif.).

The yeast was grown in minimal dextrose medium obtained from DIFCO (Detroit, Mich.), supplemented with histidine (MDH:1.34% YNB without amino acids, 4×10⁻⁵% biotin, 2% dextrose, 0.004% L-histidine) and was induced in MMH medium (minimal methanol medium supplemented with histidine: 1.34% YNB, 4×15 10⁻⁵% biotin, 1.5% methanol, 0.004% L-histidine).

### Example 2

### Construction of Expression Plasmid

This example describes construction of the expression plasmid.

Complementary DNAs (666 bp and 1332 bp of light and heavy chains, respectively) anti-dioxin genes were cloned separately into a PPICZ/α P. pastoris integrative vector with zeocin resistance gene. For the cloning, the genes were placed under the control of AOX1 promoter alongside of α-factor signal sequence using the EcoRI ends blunt-ended with T4 polymerase prior to digesting with BsmBI using methods known in the art.

The PCR primers were synthesized with a BglII site incorporated at the end of the stop codon, the product of the cDNA was cloned through BglII site ligated into a BsmBI site of the vector, resulting in the loss of both sites in the recombinant plasmid generated.

The individual recombinant plasmids were then digested with BamHI and MluI, for the recombinant containing the light chain and with BglII and MluI with the heavy chain construct. From each construct a 3134-bp fragment representing the light chain and 4030-bp fragment representing the heavy chain constructs were gel-eluted and religated to contain both the light and heavy chain genes.

The construct was introduced into E. coli XL1-Blue by electroporation, and recombinants were selected by scoring for zeocin (25 mg/ml) resistance. DNA was extracted and purified from recombinants, confirmed by colony PCR, linearized with DraI and used for transforming P. pastoris SMD1168 by electroporation using Gene Pulser (Bio-Rad, Richmond, Calif.).

Cells were regenerated in ice-cold 1 M sorbitol (1 ml) at 30° C. for 2 hours, plated in 10-100 μl portions on YEPD medium (1% yeast extract 2% peptone, 2% dextrose) containing zeocin (100 μg/ml) and incubated at 30° C. for 3-5 days. DNA manipulations were all performed using standard techniques or as recommended by the respective reagent vendors.

Recombinant colonies were each screened for the presence of genome-integrated inserts using colony-PCR, and for growth, at 30° C. for 3-4 days on Minimal Methanol (MM) medium.

### Example 3

### Expression-Screening of Transformants

This example describes procedure used for screening of transformants.

The yeast colonies, which grew on zeocin selection, were replica-plated on MM agar plates and incubated for 2 days at 30° C., colonies were covered with nitrocellulose membrane and allowed to grow further for 2-3 days at 30° C. The membranes with yeast colonies were washed 3× with TBST, blocked for 1 hour with nonfat dry milk (10%; w/v) in TBST, and incubated with Alkaline Phosphatase-conjugate (AP-goat) of goat antimouse monoclonal antibody (Boehringer Mannheim, Ind., USA) diluted 1:5000 in TBST. After 1 hour, the membranes were washed 5× with TBST and developed in the dark for 10-30 minutes at room temperature in 100 mM Tris-HCl, pH 7.5, 50 mM NaCl and MgCl₂ containing the chromogenic substrates, NBT and BCIP.

### Example 4

### DNA Sequencing

This example describes DNA sequencing protocol.

The nucleotide sequence of the constructs was determined using the chain terminating method according to PNAS (USA), 74:5463 (1977) of DNA sequencing using the ABI Taq DyeDeoxy terminator cycle sequencing kit on an automated DNA Sequencer (ABI 373A, Applied Biosystems, Foster City, Calif.). The sequences were analyzed using the ABI Prism software for sequence analysis.

### Example 5

### PCR Analysis of Expression Cassettes

This example describes PCR analysis of expression cassettes.

Genomic DNA was isolated from transformed and control (non-transformed) yeast cells, and 400 ng was tested for the presence of expression cassettes by PCR analysis using the following specific oligonucleotide primers for the light-chain gene:

Forward: 5′-GACGTCGTGATGACCCAAGCTCCA-3′ (SEQ ID NO:1)

Reverse:5′-CGCGTCTAGATCTAACACTCATTCCTGTTGAA-3′ (SEQ ID NO:2) the heavy-chain gene:

Forward: 5′-CAGGTCCAACTGCAGCAG-3′ (SEQ ID NO: 3)

Reverse: 5′-CGCGTCTAGATCTAGCATTTACCAGGAGAG-3′(SEQ ID NO: 4) the yeast AOX1 promoter

Forward: 5′-GACTGGTTCCAATTGACAAGC-3′ (SEQ ID NO: 5) Reverse: 5′-GCAAATGGCATTCTGACATCC-3′ (SEQ ID NO: 6).

Thirty-five cycles of 94° C. for 1 minute, 54° C. for 1 minute and 72° C. for 1 minute were used for PCR in a thermocycler (Model PTC150; MJ Research, Watertown, Mass.).

### Example 6

### Induction of Antibody Expression in Recombinant P. pastoris Clones

This example illustrates procedure used for inducing antibody expression in recombinant P. pastoris clones.

A transformant producing high levels of recombinant antibody was cultured overnight in BMGY broth (1.34% YNB without amino acids or ammonium sulfate, 1.0% glycerol, and 0.4 mg biotin/1) at 30° C. for 2 days with shaking at 250 rpm.

The cells were collected by centrifugation and transferred to an inducing medium (1.0% casamino acids, basal medium, trace elements, pH 5.5-6.0, 0.5% methanol and 0.004% biotin). Beginning on the second day and up to fourth day of growth, methanol was added daily to a concentration of 0.5% (v/v), to induce recombinant protein production, and the culture medium and cells were collected separately after low speed centrifugation at 4° C. and stored at 20° C. One hundred μl of a protease inhibitor cocktail (0.35 mg PMSF, 0.31 mg benzamidine, 0.2 mg aprotinin, 0.24 mg pepstain A, 0.2 mg leupeptin, 0.2 mg phenanthroline; Sigma, St. Louis, Mo.) was added to each induced culture just before harvesting. Slot blots of nitrocellulose membrane containing ten times concentrated supernatants (0.25 ml each) or cell lysates were blocked with nonfat dry milk (10%, w/v, in TBST) for 1 hour, probed with HRP-goat anti-mouse IgG, and developed using chromogenic TMB Microwell Peroxidase Substrate System (Pierce, Rockford, Ill.).

### Example 7

### Northern Blotting

This example describes conditions used for Northern blotting analysis.

Cells from 5-ml induced cultures of the yeast clones were used for total RNA extraction using a standard method. The cells, harvested by centrifugation at 1000× g for 10 minutes at 4° C. were resuspended in 400 gl TLS buffer (10 mM Tris-Cl₂ pH 7.4, 1.0% SDS, 100 mM LiCl) and were extracted with TLS buffer-saturated phenol, followed by phenol:chloroform.

Total RNA was ethanol-precipitated, washed with 70% ethanol, air-dried and resuspended in DEPC-treated TLS buffer, and electrophoresed on a 1% agarose gel containing formaldehyde (1.2%) obtained from SEAKEM GTG, Rockland, Me. RNA was transferred onto nylon membrane (Hybond-N) overnight. The membrane was hybridized overnight with α³²P-labelled light chain PCR product, followed by washings, first with 2× SSC buffer at 37° C. for 15 minutes, repeated for 20 minutes at room temperature, and finally with 1× SSC buffer at 37° C. for 15 minutes. The membrane was then air-dried and exposed to a Kodak X-Omat film for 24 hours at temperature −80° C.

### Example 8

### Antibody Expression in Transformed P. pastoris Clones

This example describes procedure used for detection of antibody expression in transformed P. pastoris clones.

A colony of a high producing transformant was cultured in BMGY broth (1.34% YNB without amino acids ammonium sulfate, 1.0% glycerol, and 0.4 mg biotin/1) overnight at 30° C. with shaking at 250 rpm for 2 days. The culture was centrifuged and transferred to an inducing medium made up of 1.0% casamino acids, basal medium, trace elements, pH adjusted with ammonia solution to 5.5-6.0, 0.5% methanol and 0.004% biotin). Cells were allowed to grow further in this medium for 2-4 days at 30° C. with shaking at 250 rpm, and 100% methanol was added daily to a concentration of 0.5%.

After this period of induction, also described in Example 5, and secretion of protein, cells were harvested by centrifugation at 2500 rpm at 4° C., supernatant was decanted and stored at −20° C. until needed. 0.5 ml of each culture medium was dispensed into wells of slot blot apparatus containing nitrocellulose membrane, and vacuum applied until liquid drained out completely. The membrane was blocked with nonfat powdered milk made up in TBST buffer to 10%. After one hour of blocking, the blot was developed with anti-mouse antibody conjugated with horse radish peroxidase (HRP) and developed using TMB (Pierce, Rockford, Ill.).

Proteins in the culture medium of a positive colony were precipitated with ice-cold acetone concentrated for brief centrifugation (10 minutes) and dried under vacuum. The concentrated proteins were then dissolved in 30 μl sample buffer and 10 μl of each resuspended sample was loaded into wells of a 10% SDS-polyarylamide gel. Gel was stained with coomassie brilliant blue R250 for 30 minutes and destained with acetic acid-methanol-water mixture.

### Example 9

### Western Immunoblot Analysis

This example describes SDS-PAGE analysis and Western blot analysis.

The culture medium (250 μl) containing recombinant anti-dioxin antibody was concentrated 25-fold by precipitation with acetone, and 10 μl of the lysates obtained by dissolving each cell pellet in 30 μl sample buffer were separately resolved by 10% SDS-PAGE (2 hours at 100 V) and were either stained with Coomassie brilliant blue R250 (Sigma, St. Louis, Mo.) for 30 minutes and destained using acetic acid/methanol/water (5:25:70), or were electroblotted onto a PVDF membrane (Millipore, Bedford, Mass.), using Tris-glycine-SDS buffer. The blot was blocked with non-fat milk (5%, w/v, in TBST, pH 8.0), incubated with AP-goat anti-mouse IgG (1:5000, in TBST, pH 8.0) for 1 hour and was washed 4 times in TBST with vigorous shaking. After 25 minutes, it was developed in the dark, with BCIP and NBT, in AP buffer, pH 7.5, at room temperature.

### Example 10

### ELISA Assay

This example describes functional assay using ELISA, for assaying the hapten-binding activity of the recombinant antibody (Antibodies: A Laboratory Manual, Eds. Harlow, E. and Lane, D., Cold Spring Harbor Laboratory (1988)).

Microplates were coated with 100 μl well⁻¹ of 50 mM bicarbonate buffer, pH 9.6, 10-50 ng of hapten for 3 hours at 37° C., followed by overnight incubation at 4° C. The plates were then equilibrated at room temperature for 45 minutes, and blocking for non-specific binding was performed for 1 hour at 37° C. with Tris-buffered saline (TBS; 10 mM Tris-Cl, pH 7.5, 150 mM NaCl) containing 1% (w/v) TBST and 3% BSA.

Plates were washed 3×0 with TBST and incubated for 2 hours at room temperature with HRP-goat anti-mouse IgG. The plates were again washed 4× with TBST, and the HRP activity of the bound antibody was assayed for 10 minutes at 37° C., using the substrates: O-phenylene diamine (OPD) (30 mg) and H₂O₂ (30 μl), dissolved in 75 ml phosphate-citrate buffer (0.1 M citric acid, 0.2 M Na₂HPO₄, pH 5.0). The reaction was terminated by the addition of 1 M H₂SO₄ (100 μl), and the plates were read at 492 nm, using the UVmax kinetic microplate reader (Molecular Devices Corp., Menlo Park, Calif.).

All samples were normalized against a sample blank, and read and analyzed using the software package, Softmax (Molecular Devices, Menlo Park, Calif.).

### Example 11

### Slot-Immunoblot Analysis

This example describes slot-immunoblot analysis of hapten-binding activity of recombinant antibody.

The ability of the recombinant antibody to bind its cognate hapten was assayed using slot-blot approach. The hapten (BSA-dioxin, diluted 1:3,600, to 250 mg/slot⁻¹) was spotted in 5 or 10 μl volumes onto nitrocellulose membrane, using a slot-blot apparatus (Schleicher & Schuell, Keene, N.H.).

The blot was allowed to air-dry, blocked with non-fat powdered milk (10%, w/v, in Tris-NaCl-Tween 20) at room temperature for 1 hour, and was then incubated with mouse anti-c-myc antibody (diluted 1:5000 in TBST buffer) for 1 hour at room temperature. The blot was then washed 4× with TBST buffer, incubated with AP-goat anti-mouse IgG (1:5000, in TBST, pH 8.0) for 1 hour at room temperature, again washed with vigorous shaking 5× over a period of 30 minutes, and developed in the dark, with BCIP and NBT, in AP buffer, pH 7.5 at room temperature.

### Example 12

### Kinetics of Recombinant Antibody Synthesis

This example illustrates kinetics of recombinant antibody synthesis and secretion.

The kinetics of antibody production and secretion were followed using slot-immunoblots. Single colonies of the clones 11505-1 and 112535-1 and the vector control grown in BMGY broth were induced in MMH medium, and culture media and cells were collected at 12-hour intervals between 0 and 120 hours. Culture media (500 μl slot⁻¹) and cell lysates (10 μl slot⁻¹) were slot-blotted onto a nitrocellulose membrane, blocked and probed with AP-goat anti-mouse IgG as described for the Western immunoblot and in Example 10.

What is claimed:

1. A method for a large-scale production of antigen-specific intact antibody, said method comprising steps:
(a) isolating cDNA, mRNA or genomic DNA of genes for antibody light and heavy chains and assembling the antibody genes into expression cassettes containing the cDNA;
(b) preparing a recombinant P. pastoris yeast expression vector;
(c) constructing a recombinant P. pastoris yeast expression plasmid containing the expression cassettes of cDNA of the light and heavy chain genes encoding the antibody;
(d) cloning the antibody expression cassettes into the P. pastoris expression vector to generate recombinant plasmid;
(e) transforming Saccharomyces cerevisiae with the recombinant plasmid by placing said expression cassettes under the control of the AOX1 promoter fused to a Saccharomyces cerevisiae α-mating factor signal sequence;
(f) amplifying and isolating the recombinant plasmid;
(g) preparing and transforming P. pastoris with BglII, NotI, SacI, SalI or Stul-linearized recombinant plasmid replacing the yeast chromosomal AOX1 sequence with AOX1-antibody gene cassettes of the recombinant plasmid;
(h) selectively growing the recombinants;
(i) screening yeast transformation colonies for a recombinant antibody expression;
(j) analyzing putative positive yeast clones for chromosomal integrates of the expression cassettes of heavy and light chain cDNAs;
(k) confirming the integrity of the DNA insert or junction sequence;
(1) inducing the recombinant antibody expression;
(m) confirming the intactness of the expression cassettes inserts with PCR and Northern blot analysis;
(n) detecting the presence of the recombinant antibody by Western blot; and
(o) testing the recombinant antibody for specific antigen-antibody binding.

2. The method of claim 1 wherein the antibody genes are assembled into the expression cassettes by subcloning the antibody light and heavy chain cDNA in tandem EcoRI-BglII/BsmBI fragments flanked by a P. pastoris signal sequence, preceded by a P. pastoris promoter at the 5′ terminus and a P. pastoris yeast transcription termination sequence at the 3′-terminus.

3. The method of claim 2 wherein the signal sequence is α-factor and wherein the promoter is AOX1-P.

4. The method of claim 3 wherein the yeast expression vector is pPICZα.

5. The method of claim 4 wherein the yeast expression vector is prepared by restriction digestion with EcoRI and BamHI.

6. The method of claim 5 wherein the recombinant plasmid is pPICZαLH.

7. The method of claim 6 wherein the recombinant expression plasmid pPICZαLH is constructed by cloning the antibody genes expression cassettes into the P. pastoris expression vector.

8. The method of claim 7 wherein the replacement of the yeast chromosomal AOX1 with AOX1-antibody gene cassettes is by homologous recombination replacement.

9. The method of claim 8 wherein the selective growth of the recombinants is performed on a medium containing zeocin.

10. The method of claim 9 wherein the selective growth of the recombinants is performed on a medium containing g418, trimethoprin, or a compound that limits the growth of wild type P. pastoris.

11. The method of claim 10 wherein the screening of transformed colonies is by colony-immunoblotting.

12. The method of claim 11 wherein the screening is by PCR or by restriction analysis.

13. The method of claim 12 wherein the integrity of the DNA inserts or junction sequence is confirmed by nucleotide sequence analysis.

14. Intact antigen-specific antibodies produced by P. pastoris transformed with mouse, humanized mouse or human immunoglobulin genes, said antibody produced by the process comprising steps:
(a) isolating cDNA, mRNA or genomic DNA of genes for antibody light and heavy chains and assembling the antibody genes into expression cassettes containing the cDNA;
(b) preparing a recombinant P. pastoris yeast expression vector;
(c) constructing a recombinant P. pastoris yeast expression plasmid containing the expression cassettes of cDNA of the light and heavy chain genes encoding the antibody;
(d) cloning the antibody expression cassettes into the P. pastoris expression vector to generate recombinant plasmid;
(e) transforming Saccharomyces cerevisiae with the recombinant plasmid by placing said expression cassettes under the control of the AOX1 promoter fused to a Saccharomyces cerevisiae α-mating factor signal sequence;
(f) amplifying and isolating the recombinant plasmid;
(g) preparing and transforming P. pastoris with BglII, NotI, SacI, SalI or Stul-linearized recombinant plasmid replacing the yeast chromosomal AOX1 sequence with AOX1-antibody gene cassettes of the recombinant plasmid;
(h) selectively growing the recombinants;
(i) screening yeast transformation colonies for a recombinant antibody expression;
(j) analyzing putative positive yeast clones for chromosomal integrates of the expression cassettes of heavy and light chain cDNAs;
(k) confirming the integrity of the DNA insert or junction sequence;
(1) inducing the recombinant antibody expression;
(m) confirming the intactness of the expression cassettes inserts with PCR and Northern blot analysis;
(n) detecting the presence of the recombinant antibody by Western blot; and
(o) testing the recombinant antibody for specific antigen-antibody binding and intactness.

15. The antibody of claim 14 wherein the antibody genes are assembled into the expression cassettes by subcloning the antibody light and heavy chain cDNA in tandem EcoRI-BglII/BsmBI fragments flanked by a P. pastoris signal sequence, preceded by a P. pastoris promoter at the 5′terminus and a P. pastoris yeast transcription termination sequence at the 3′-terminus.

16. The antibody of claim 15 produced by P. pastoris transformed with human immunoglobulin genes.

17. The antibody of claim 15 produced by P. pastoris transformed with humanized mouse immunoglobulin genes.

18. The antibody of claim 15 produced by P. pastoris transformed with mammalian or mouse immunoglobulin genes.

19. A recombinant P. pastoris yeast expression vector containing dual expression cassettes, each carrying a cDNA copy of immunoglobulin light and heavy chain.

20. An expression system comprising P. pastoris transformed with antibody genes for production of a recombinant antigen-specific intact antibody.

21. P. pastoris yeast transformed with expression cassettes carrying a cDNA copy of immunoglobulin heavy and light chain suitable for large-scale production of intact antibodies.
